(19) United States
(12) Patent Application Publication
(10) Pub. No.: US 2021/0177313 A1
(43) Pub. Date: Jun. 17, 2021

(54) CONTINUOUS HEALTH MONITORING SYSTEM

(71) Applicant: Indigo Diabetes NV, Gent (BE)

(72) Inventors: Juan Sebastian ORDONEZ ORELLANA, Gent (BE); Danaë DELBEKE, Gentbrugge (BE)

(21) Appl. No.: 17/183,921

(22) Filed: Feb. 24, 2021

Related U.S. Application Data
(63) Continuation-in-part of application No. 16/639,768, filed on Feb. 18, 2020.

(30) Foreign Application Priority Data
Aug. 18, 2017 (EP) 17186763.3

(51) Int. Cl. A61B 5/1473; A61B 5/145; A61B 5/00; A61B 5/053; A61B 5/16; G16H 40/60

(52) U.S. Cl. CPC A61B 5/1473; A61B 5/14532; A61B 5/14539; A61B 5/746; A61B 5/6802; A61B 5/7475; A61B 5/024; A61B 5/4875; A61B 5/4866; A61B 5/7278; A61B 5/14546; A61B 5/165; G16H 40/60; A61B 5/053

(57) ABSTRACT

A health monitoring system for monitoring health of a living creature is described. The health monitoring system comprises an implantable sensor for simultaneously measuring a plurality of metabolism analytes in bodily tissue or bodily fluids of the living creature, thus obtaining a metabolic fingerprint for a living creature comprising concentration information of the plurality of metabolism analytes at a given moment in time. The health monitoring system furthermore comprises a processor programmed for determining for each of the plurality of metabolism analyte whether the obtained concentration information is within a predetermined range.

This application is a continuation-in-part application claiming benefit of U.S. patent application Ser. No. 16/639,768, filed Feb. 18, 2020, which is an nationalization of international application PCT/EP2018/072317, filed Aug. 17, 2018, which claims the benefit of EP application 17186763.3, filed Aug. 18, 2017, the entirety of said applications being incorporated herein by reference.

### FIELD OF THE INVENTION

The invention relates to the field of health monitoring. More particularly, the present invention relates to systems and methods for health monitoring using an implantable device based on simultaneous detection of a plurality of metabolism analytes.

### BACKGROUND OF THE INVENTION

Personal health monitoring gains in importance, in view of the increased attention of people for personal health. Conventionally, one well-established way of monitoring personal health is through regular consults with a medical practitioner. Often during such a consult, blood analysis is performed for checking certain analytes. The concentration of analytes in bodily fluids may provide information regarding health.

A personal health monitoring system is for example known from U.S. Pat. No. 8,718,943, which describes a health-monitoring device which assesses the health of a user based on levels of two analytes in a biological fluid. A first analyte that is utilized to assess a user's health is a fat metabolism analyte, such as ketones, free fatty acids and glycerol, which is indicative of fat metabolism. A second analyte that is utilized is a glucose metabolism analyte, such as glucose. The levels of the two analytes are used to assess insulin sensitivity, to detect both recent hypoglycemia and the cause of high glucose levels, and/or to guide therapeutic intervention. The dual analyte model calculates a discrepancy between an actual insulin activity level and a theoretical insulin activity level.

The need for consults with a medical practitioner is often considered cumbersome. Therefore, a number of personal health monitoring systems have been developed in the past to assist in monitoring health of a user.

US 2017/0095216 A1 describes a wrist-worn biowatch providing various health monitoring functions such as blood glucose level monitoring, blood pressure detection, pulse monitoring, heart stop detection, oxygen saturation monitoring, and Ketoacidosis detection. The biowatch is actively monitoring the wellness data of its wearer, and adapted to alert the user and medical professionals if such wellness data veers outside normal ranges or acceptable trends.

### SUMMARY OF THE INVENTION

It is an object of the present invention to provide methods and systems allowing continuously monitoring of the health of a living creature. It is an advantage according to embodiments of the present invention that such health monitoring can be performed in a real individualized and personal manner. It is an advantage of embodiments of the present invention that health monitoring can be based on a metabolic fingerprint, providing information regarding metabolism analytes in the human body.

It is an advantage of embodiments of the present invention that a personal health monitoring system is provided allowing to have “the doctor's visit in your pocket”. It thereby is an advantage that the special conditions of a doctor's visit and the affected results thereby are exchanged for a real continuous monitor that measures in real environmental situations, under real conditions of the activation of the metabolism and of the autonomous nervous system controls.

It is also an object of the present invention to provide a platform for assisting in monitoring health of living creatures and/or in assisting in assessment of the metabolic impact of a selected lifestyle, drug use, etc.

It is an advantage of embodiments of the present invention that methods and systems are provided allowing an accurate evaluation of a plurality of metabolism analytes in a living creature by monitoring thereof and by providing data being captured at the same moment in time, i.e. by providing paired data.

The object and optionally also advantages are obtained by methods and systems according to aspects of the present invention.

The present invention relates to a health monitoring system for monitoring health of a living creature, the health monitoring system comprising

an implantable sensor for simultaneously measuring a plurality of metabolism analytes in bodily tissue or bodily fluids of the living creature, thus obtaining a metabolic fingerprint for a living creature comprising concentration information of the plurality of metabolism analytes at any given moment in time, and

a processor programmed for determining for each of the plurality of metabolism analyte whether the obtained concentration information is within a predetermined range.

The processor furthermore may be programmed for deriving from said determination a homeostatic and/or alleostatic condition of the living creature.

The plurality of metabolism analytes may comprise at least three different metabolism analytes.

The plurality of metabolism analytes may be a selection of two or more of glucose, ketones, lactate, lactic acid, acetic acid, nitrite, creatinine, hormones, vitamine B11, vitamine B12, acetate, propionic acid, propionate, butyric acid, butyrate, phenyl sulfate, urea, pyruvate, ethanol, sugar, pH, uric acid, lactate dehydrogenase, fasting glucose, sodium, potassium or cholesterol.

The health monitoring system may be adapted for obtaining information regarding the user's medical condition.

The health monitoring system furthermore may comprise an output port for providing an output to a user, a medical practitioner or a medical team and the health monitoring system may be programmed for providing personalised recommendations to the user through the output port.

The system may be configured to measure the different metabolism analytes at different moments in time.

The system may be configured to, based on said determining for each of the plurality of metabolism analytes whether the obtained concentration information is within a predetermined range, trigger additional measurements of one or more metabolism analytes or to alter a frequency of measurement of one or more metabolism analytes.

The system may be configured for, based on said determining for each of the plurality of metabolism analytes whether the obtained concentration information is within a predetermined range, providing the user with a warning for consulting a medical practitioner or for providing a warning to a medical practitioner of the user.

The health monitoring system may further comprise an input port for obtaining information regarding one or more physiological parameters of the living creature at the time of measurement of the plurality of metabolism analytes in bodily tissue or bodily fluids of the living creature.

The input port of the health monitoring system may be configured for receiving the information regarding one or more physiological parameters from a wearable, worn by the living creature.

The health monitoring system may be configured for receiving user input from a user, optionally through a graphical user interface.

The health monitoring system may be programmed for prompting the user for input when one or more metabolism analyte concentrations outside the predetermined range is detected.

The health monitoring system may be configured for deriving from the physiological parameters a stress condition and for identifying that the measured metabolism analyte concentrations are determined under the stress condition.

The health monitoring system may comprise an input port for obtaining information regarding one or more of the user's location, hobbies, ethnic background, age, gender, socio-economical status, etc.

The processor may be configured for comparing with predetermined ranges for the metabolism analyte concentrations, whereby the predetermined ranges are based on previously measured metabolism analyte concentrations from the living creature.

The processor may be configured for comparing with predetermined ranges for the metabolism analyte concentrations, whereby the predetermined ranges are based on previously measured metabolism analyte concentrations obtained from a group of living creatures.

The system may be adapted for obtaining information regarding to any of gender, age, ethnicity or body mass index and wherein the processor furthermore is configured for comparing with predetermined ranges that are based on previously measured metabolism analyte concentrations obtained from a group of living creatures having a same gender, age, etc.

The implantable sensor may comprise an optical sensor configured for spectroscopic measurement of the plurality of metabolism analytes in bodily tissue or bodily fluids.

The system furthermore may comprise an impedance spectroscopy sensor for deriving a hydration status. In one aspect the present invention also relates to a health monitoring platform, the health monitoring platform comprising a plurality of health monitoring systems as described above for use by a plurality of individual users and further comprising a central processing unit for processing metabolic fingerprint information obtained from the plurality of health monitoring systems.

In one aspect, the present invention also relates to a method for obtaining personal health information for an individual, the method comprising

simultaneously measuring a plurality of metabolism analytes in bodily tissue or bodily fluids of the living creature, thus obtaining a metabolic fingerprint for a living creature comprising concentration information of the plurality of metabolism analytes at any given moment in time, and

determining for each of the plurality of metabolism analyte whether the obtained concentration information is within a predetermined range.

In other aspects, it is an aim of some embodiments of the present invention to provide a personal health monitoring system, comprising an implantable sensor and a monitoring device, and capable of generating an improved personal health profile from the collected sensor data. It is also an aim of some embodiments of the present invention to provide a multiple user health monitoring system with improved capabilities of monitoring health conditions of user groups. It is also an aim of some embodiments of the present invention to provide an efficient method for monitoring biological parameters of at least one user. It is also an aim of some embodiments of the present invention to provide to the user personalized behavioural, lifestyle and therapeutic advice and/or interventions, based on the monitoring of those biological parameters.

In one aspect, some embodiments of the present invention provide a personal health monitoring system, comprising an implantable sensor and a monitoring device. The implantable sensor comprises sensing means for sensing biological parameters in bodily fluids of a user and a first wireless transceiver for transmitting sensor data containing data points which are provided by said sensing means upon sensing said biological parameters. The sensed biological parameters comprise at least a glucose concentration and a ketone bodies concentration in said bodily fluids, such that said sensor data comprises at least glucose concentration data points and ketone bodies concentration data points. The monitoring device comprises a second wireless transceiver for communicating with said first wireless transceiver to receive said sensor data and processing means for processing said sensor data, wherein said processing means is equipped with an algorithm which is executable on said processing means and which, when executed, is provided for performing the following steps: determining first trends in said glucose concentration data points and second trends in said ketone bodies concentration data points; and generating a personal health profile of the user.

The personal health monitoring system according to some embodiments of the invention senses and processes at least glucose and ketone bodies concentrations in bodily fluids.

The personal health monitoring system according to some embodiments of the invention uses the algorithm to analyse the sensor data. In preferred embodiments according to the invention, the algorithm is provided for detecting trends in sets of data points, and correlating trends of different sets of data points with each other. In this way, trends are detectable which are personal, i.e. specific to the user carrying the implanted sensor. Likewise, correlations between the trends in different biological parameters can be determined in a personal way, such as for example a normal evolution of glucose and ketones concentration for that user during the night or a normal evolution of glucose and ketones concentration for that user after a certain meal or a certain activity, etc. In this way, the personal health monitoring system according to the invention may be capable of “learning” for example which are normal evolutions of the biological parameters of the user and which are not normal and include this information in the personal health profile which it generates for the user. This information can then be further used by the system to for example make predictions, issue warnings, etc. In preferred embodiments according to the invention the algorithm of the monitoring device is provided for performing the following steps: determining first trends in said glucose concentration data points and second trends in said ketone bodies concentration data points; detecting first user dependent correlations between said first trends and said second trends, and generating a personal health profile of the user based on said first user dependent correlations.

In some embodiments according to the invention, the personal health monitoring system may be provided for sensing and/or processing at least one of the following additional parameters: heart rate, body temperature, urea, lactate, pH, fructosamine, oxaloacetate and/or hydration level. It has been found that by taking one or more of these parameters into account, detecting trends and possibly correlations with the trends in other biological parameters, a further improved personal health profile may be achieved.

In some embodiments according to the invention, the monitoring device may comprise a display for displaying the personal health profile. In embodiments, the monitoring device may be a mobile terminal such as a smart phone, tablet, smart watch or other wearable device. In embodiments, the monitoring device may be a dedicated monitoring device which is specifically designed for the purpose of communicating with the implantable sensor and generating the personal health profile. In embodiments the monitoring device may be provided for generating and/or communicating to the user personalized behavioural, life-style and therapeutic suggestions and actions, based on the monitoring of biological parameters. In embodiments, the monitoring device may be provided for generating instructions for a controller of an insulin pump or may form part of an insulin pumping device. In embodiments, the monitoring device may be an ensemble of one or more devices.

In some embodiments according to the invention, the algorithm of the monitoring device may be provided for combining the sensor data with metadata (such as location data, calories intake data, activity data, agenda information and/or user habit information) upon generating the personal health profile. In case a mobile terminal is used as monitoring device, any metadata generated by means of applications running on the mobile terminal itself may be used for this purpose.

The implantable sensor may be capable of continuous monitoring of biological parameters. The term ‘continuous’ or ‘continuously’ in relation to the invention should be construed as meaning ‘regularly without requiring regular user intervention’, the sampling rate can be a fixed number of measurements per time frame or varied by an integrated controller. In embodiments according to the invention, the implantable sensor may comprise an integrated controller which is provided for controlling the sensing means at a variable sampling rate. In embodiments, the integrated controller may be provided for detecting a variability level in said sensor data and adapting said variable sampling rate according to said detected variability level, for example by reducing the sample rate if a low variability level (beneath a certain threshold) is detected. In other embodiments, the sampling rate may also be controlled by the monitoring device. By reducing the sampling rate, for example when it is expected that the sensor data will not vary much over a longer period of time, energy consumption of the sensing means of the implanted sensor can be reduced and battery life can possibly be extended.

In some embodiments according to the invention, the implantable sensor may comprise a rechargeable battery and/or components for wireless energy transfer, such that recharging can occur without having to remove the (implanted) sensor.

Some embodiments of the invention provide a multiple user health monitoring system which comprises a plurality of the personal health monitoring systems as described above. The multiple user health monitoring system comprises a remote server system which is provided for collecting the personal health profiles generated by the plurality of personal health monitoring systems. As a result of the self-learning capabilities of the individual personal health monitoring systems, the collected information can efficiently be used to generate e.g. reports, statistics, etc. of user groups.

Some embodiments of the invention provide a method for monitoring the biological parameters of at least one user. The method, and embodiments thereof, comprise substantially the steps as have already been described above in relation to the personal health monitoring system according to the invention.

Particular and preferred aspects of the invention are set out in the accompanying independent and dependent claims. Features from the dependent claims may be combined with features of the independent claims and with features of other dependent claims as appropriate and not merely as explicitly set out in the claims.

These and other aspects of the invention will be apparent from and elucidated with reference to the embodiment(s) described hereinafter.

### BRIEF DESCRIPTION OF THE DRAWINGS

The present invention will be discussed in more detail below, with reference to the attached drawings.

FIG. 1 shows an embodiment of the implantable sensor comprising sensor housing 1, sensing means 2, sensor housing adapted to sensing means 3 and a wireless transceiver 4.

FIG. 2 shows an embodiment of the implantable sensor with optical sensing means 5 and optical processor 6.

FIG. 3 shows an embodiment of the implantable sensor with components for wireless energy transfer 7.

FIG. 4 shows an embodiment of the implantable sensor with components for wireless energy transfer 7 and optical sensing means 5 and optical processor 6.

FIG. 5 shows an embodiment of the implantable sensor with processing means 9.

FIG. 6 shows an embodiment of the monitoring device comprising a wireless transceiver 4, monitoring device housing 8, processing means 9 and a memory 10.

FIG. 7 shows an embodiment of the monitoring device with display 11.

FIG. 8 shows an embodiment of the monitoring device with heart rate sensor 12.

FIG. 9 shows an embodiment of the monitoring device as an ensemble of devices.

FIG. 10 shows another embodiment of the monitoring device as an ensemble of devices.

FIG. 11 shows an embodiment of the algorithm of the health monitoring system.

FIG. 12 shows another embodiment of the algorithm of the health monitoring system.

FIG. 13 shows an embodiment of the algorithm of the health monitoring system with a heart rate monitor in the monitoring device.

FIG. 14 shows another embodiment of the algorithm of the health monitoring system with a heart rate monitor in the monitoring device.

FIG. 15 shows an embodiment of the health monitoring system comprising a remote server.

FIG. 16 shows an embodiment of the interaction of monitoring devices with a remote server.

FIG. 17 shows another embodiment of the interaction of monitoring devices with a remote server.

FIG. 18 shows an embodiment of the interaction of the monitoring device with an insulin pump controller.

FIG. 19 shows a schematic overview of an embodiment of a health monitoring system according to an aspect of the present invention.

The drawings are only schematic and are non-limiting. In the drawings, the size of some of the elements may be exaggerated and not drawn on scale for illustrative purposes.

Any reference signs in the claims shall not be construed as limiting the scope.

In the different drawings, the same reference signs refer to the same or analogous elements.

### DETAILED DESCRIPTION OF ILLUSTRATIVE EMBODIMENTS

The present invention will be described with respect to particular embodiments and with reference to certain drawings, but the invention is not limited thereto but only by the claims. The drawings described are only schematic and are non-limiting. In the drawings, the size of some of the elements may be exaggerated and not drawn on scale for illustrative purposes. The dimensions and the relative dimensions do not correspond to actual reductions to practice of the invention.

Furthermore, the terms first, second and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

Moreover, the terms top, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

Where in embodiments of the present invention reference is made to a metabolic fingerprint, reference is made to concentration values of a set of metabolism analytes—as determined at a given moment in time. A metabolic fingerprint typically may comprise a plurality of metabolism analytes, such as for example two or more metabolism analytes or three or more metabolism analytes. Depending on the metabolism analytes enclosed in the metabolic fingerprint, the metabolic fingerprint may be dedicated for identifying information regarding particular diseases or conditions, or a set of diseases or conditions. In some embodiments of the present invention, the metabolic fingerprint may comprise a large number of metabolism analytes which may provide information regarding different conditions, disorders or diseases. It is an advantage of embodiments of the present invention that the metabolic fingerprint can easily be combined with temperature information.

Where in embodiments of the present invention reference is made to a homeostatic condition, reference is made to the tendency of having—at different states—a relative stable equilibrium of metabolism analytes. Where the indication “at different states” is given, reference is made to the fact that for example a stable equilibrium during the day can be different from a stable equilibrium during the night or that for example a stable equilibrium during summer can be different from a stable equilibrium during the winter.

Where in embodiments of the present invention reference is made to an alleostatic condition, reference is made to the tendency of maintaining—within a dynamic range—a state of internal, physiological equilibrium of metabolism analytes, in response to actual or perceived environmental and psychological stressors.

Where in embodiments of the present invention reference is made to an implantable sensor, reference is made to a sensor capable of in vivo measurements of one or more biological parameters in a living creature, e.g. in an animal or human. The implantable sensor may be located subcutaneous, intramuscular, intravascular, ocular such as in or attached to the cornea, in or attached to an organ, an oral space, a brain or a bone cavity. In an advantageous embodiment, the implantable sensor is a subcutaneous sensor. In a first aspect, the present invention relates to a health monitoring system for monitoring health of a living creature, an exemplary embodiment thereof being shown in FIG. 19. The health monitoring system 100 comprises an implantable sensor 110 for simultaneously measuring a plurality of metabolism analytes in bodily tissue or bodily fluids of the living creature, thus obtaining a metabolic fingerprint for a living creature comprising concentration information of the plurality of metabolism analytes at any given moment in time, and a processor 120 programmed for determining for each of the plurality of metabolism analyte whether the obtained concentration information is within a predetermined range.

By way of illustration, embodiments of the present invention not being limited thereby, standard and optional components of the health monitoring system are described in more detail.

According to embodiments of the present invention, the health monitoring system comprises an implantable sensor that is configured for simultaneously measuring a plurality of metabolism analytes in bodily tissue or bodily fluids of the living creature, thus obtaining a metabolic fingerprint for a living creature comprising concentration information of the plurality of metabolism analytes at any given moment in time.

The health monitoring system may be a system for monitoring health of a living creature by performing measurements continuously, at predetermined moments in time, at moments in time triggered by events such as for example based on an environmental and/or lifestyle driven input such as for example driven by food intake or any other incoming information such as for example sound, tactile, smell, heat or movement. Measurements may be performed at any desired moment in time, etc. It is an advantage of embodiments of the present invention that there is no need for manual intervention, i.e. measurements can be performed in an automated way and automatically, due to the fact that measurements are based on an implantable sensor. It is an advantage of embodiments of the present invention that monitoring health can be performed without the need for consulting a medical practitioner at regular moment in times.

It is an advantage of embodiments of the present invention that measurements are performed in situ/in vivo, allowing for measuring and monitoring in the real environment, i.e. without the need for determining values of e.g. metabolism analytes on samples that are first extracted from the living creature. Extraction from the living creature typically may result in alteration of the samples and therefore may result in deviations of the measured metabolism analytes compared to their in vivo presence.

The implantable sensor of the health monitoring system according to an exemplary embodiment of the present invention typically may comprise a sensor housing, a sensing device for sensing a plurality of metabolism analytes in bodily tissue or bodily fluids. The implantable sensor also typically may comprise a wireless transceiver for transmitting sensor data containing data points which are provided by said sensing means upon sensing biological parameters. The implantable sensor may be capable of performing a reagent-free analysis method. In some embodiments, the implantable sensor may comprise biocompatible packaging in order to avoid and/or minimize the risk of bio-fouling. The implantable sensor may comprise a battery, a rechargeable battery, capacitors and/or components for wireless energy transfer. The system may be equipped with components for wireless energy transfer. In particular embodiments, the system may be equipped with a rechargeable battery such as for example with a solid state battery.

The implantable sensor may comprise an optical sensing device configured for spectroscopic measurement of the plurality of metabolism analytes in bodily tissue or bodily fluids. It is an advantage of embodiments that spectroscopic measurements allows for detection of different metabolism analytes, e.g. based on detection of absorption in different absorption bands.

It is an advantage of embodiments of the present invention that optical spectroscopy based on an implantable sensor allows for accurate determination of metabolite concentrations. The implantable sensor may for example be a semiconductor-based photonics integrated circuit sensor, such as for example a silicon-based photonics integrated circuit sensor as for example described in European patent application EP10760633.7 although embodiments are not limited thereto. It is an advantage of embodiments of the present invention that a miniaturised sensing device can be used that is at the same time small in footprint, thus providing good implantability conditions, and allows for accurate concentration determination of metabolites. It is an advantage of embodiments of the present invention that optical spectroscopy of metabolites in bodily tissue or bodily fluids based on an implantable sensor allows for detection of small concentration variations, thus resulting in accurate determination of metabolite concentration information.

In an advantageous embodiment, the sensing device also may allow for determining body temperature from the measurement. Alternatively, or in addition thereto a dedicated temperature sensing device may be included in the implantable sensor.

It is an advantage of embodiments of the present invention that measurements can be performed at very short timescale, thus allowing detection of metabolism analyte concentration variations at very short timescale, such as for example on a timescale of less than 5 minutes, e.g. in some embodiments a timescale of less than one minute, e.g. in some embodiments within seconds or parts of seconds.

It is an advantage of embodiments of the present invention that also middle- and long-term variations can be identified, e.g. variations on a timescale of hours, days or weeks. Furthermore, some variations may be time-of-year dependent and the health monitoring system also may identify such variations, e.g. by comparison of results month to month.

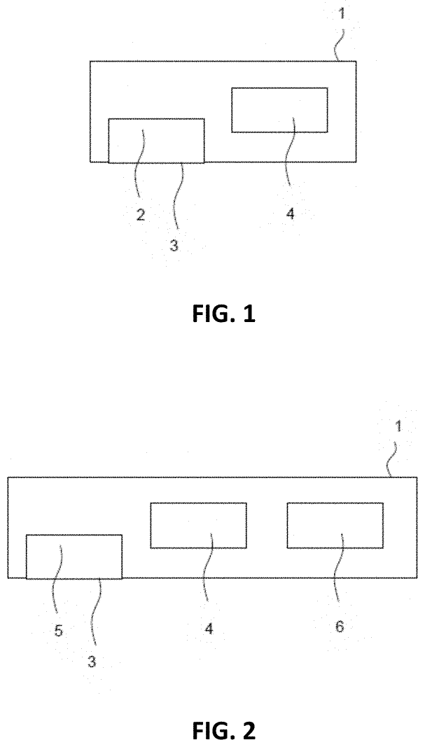

According to some embodiments, the implantable sensor may correspond with an implantable sensor as shown and described in FIG. 1 to FIG. 5, but whereby the implantable sensor is particularly adapted for simultaneously obtaining a plurality of metabolites.

The system furthermore may comprise an impedance spectroscopy sensing device for deriving a hydration status. The system may be configured for measuring a physiological ionic status. Based on impedance spectroscopy, changes in the physiological ionic status in interstitial fluids can be determined, allowing evaluation of a hydration status of a user. Impedance spectroscopy analysis thereby may be based on e.g. Nyquist/Warburg plots of the derived information.

In some embodiments, also other types of sensing devices may be included in the implantable sensor such as for example an electrochemical sensing device, an enzymatic sensing device, an electronic impedance sensing device, a resonant electronic circuit sensing device, or an electrical sensing device such as a resistive sensing device. Also other type of sensing devices may be included such as for example accelerometers, orientation sensing devices, pH sensing devices, electrical activity (e.g. conductivity) sensing devices, etc. It is an advantage of embodiments of the present invention that different metabolites can be determined simultaneously based on detection in bodily tissue or fluids, thus allowing a more accurate evaluation of a unique health condition of a living creature. Whereas often statistically driven information may be used for controlling and/or monitoring health, in practice effects may in principle be personal and therefore unique to certain living creatures. It is an advantage of embodiments of the present invention that it allows identifying whether effects—which statistically may occur for a population—are present for the individual or that it allows identifying the conditions under which such effects occur for an individual. It thus is an advantage of embodiments of the present invention that a real personalised health monitoring system is obtained.

It is an advantage of embodiments of the present invention that the health monitoring system can be used as a tool for investigating whether certain effects occur for individuals or for given populations and for investigating for example the effect of food, lifestyle choices or drug uptake on the individual or on a given population.

It is an advantage of embodiments of the present invention that mutual influences or combined effects can be accurately determined since the obtained concentration information stems guaranteed from the same moment in time, i.e. embodiments allow for obtaining paired data. The obtained data does not need to comprise or require contextual data, although embodiments of the present invention are not limited thereto and, as will be described below, contextual data may be provided, e.g. from manual user input, through application programs on computerised devices of the user or in any other way.

According to embodiments of the present invention, the health monitoring system also may comprise a processor programmed for determining for each of the plurality of metabolism analyte whether the obtained concentration information is within a predetermined range. The processor may be any suitable type of processor for processing data as known in the field, such as for example a general-purpose processor or a specific processor, but according to embodiments of the present invention, the processor is programmed for determining for each of the plurality of metabolism analyte whether the obtained concentration information is within a predetermined range. Such ranges may be personalised ranges determined for the individual, may be ranges based on statistical information for specific groups of users, may be ranges determined based on particular physiological parameters of the users, and alike, as will be further described below.

The processor can be for example implemented on the implantable sensor sending the processed data wireless to an external device. Such an external device may be a dedicated device or a mobile device, such as for example a smartphone, a watch, a tablet or a laptop, or it may for example be a computer or may be a cloud based storage device. Alternatively, the processor can also be implemented or part of an external device such as for example a dedicated device or a mobile device, such as for example a smartphone, a watch, a tablet or a laptop, a computer or a cloud based storage device. Such an external device typically also comprises a wireless transceiver for receiving the raw or processed data. Data communication between the sensing device and the processor may be performed based on any suitable protocol. Data communication between the implantable sensor and the external device may be established using any suitable protocol, as known in the field.

By way of illustration, embodiments of the present invention not being limited thereto, external devices as can be used in embodiments of the present aspect may be as shown and described in FIG. 6 to FIG. 10, but wherein the external device is arranged for obtaining processed data comprises information regarding whether the metabolism analyte concentrations are within predetermined ranges, as indicated above, or wherein a processor is present on the external device is programmed for such processing.

The processing also may be split over different processors which may be implemented on the implantable device, on an external device or splitted over the implantable sensor and the external device. The processing may in some embodiments be splitted into cloud-base processing.

The processor may be programmed for deriving from said determination a homeostatic and/or alleostatic condition of the living creature. It is an advantage of embodiments of the present invention that a momentaneous evaluation of metabolites in the bodily tissue or bodily fluids of the living creature allows for a good evaluation of a health condition of the living creature.

The processing may be based on a predetermined algorithm. In some embodiments, processing may make use of artificial intelligence, neural networks, etc.

The processor may be configured for comparing with predetermined ranges for the metabolism analyte concentrations, whereby the predetermined ranges are based on previously measured metabolism analyte concentrations from the living creature.

The predetermined range thus can be uniquely identified for each user. Again in this way, a health monitoring system is provided that really is designed for the individual using the system.

The processor may be configured for comparing with predetermined ranges for the metabolism analyte concentrations, whereby the predetermined ranges are based on previously measured metabolism analyte concentrations obtained from a group of living creatures.

The predetermined range can in some embodiments be based on statistically-pooled health indications.

The system may be adapted for obtaining information regarding to, for example, any of gender, age, ethnicity or body mass index and wherein the processor furthermore is configured for comparing with predetermined ranges that are based on previously measured metabolism analyte concentrations obtained from a group of living creatures having a same gender, age, etc.

It is an advantage of embodiments of the present invention that the health monitoring may allow for identifying risks to certain conditions or diseases or to better manage conditions or diseases e.g. by providing recommendations for controlling lifestyle, dietary conditions or controlling personal drug uptake. It also may be used for identifying a personalised and unique response to dietary conditions, lifestyle decisions or personal drug uptake.

The plurality of metabolism analytes may comprise at least three different metabolism analytes. It is an advantage of embodiments of the present invention that information regarding more than two, e.g. three or more metabolism analytes can be determined, thus allowing to provide good information regarding a health condition of the living creature, e.g. of a homeostatic and/or alleostatic condition of the living creature homeostatic and/or alleostatic condition of the living creature.

The plurality of metabolism analytes may be a selection of two or more of glucose, ketones, lactate, lactic acid, hormones, acetic acid, nitrite, creatinine, vitamine B11, vitamine B12, acetate, propionic acid, propionate, butyric acid, butyrate, phenyl sulfate, urea, pyruvate, ethanol, sugar, pH, uric acid, lactate dehydrogenase, fasting glucose, sodium, potassium or cholesterol. Water and temperature also may be used as biological parameters for monitoring health.

By way of illustration, embodiments of the present invention not being limited thereto, some examples of the use of metabolic fingerprints will be described below.

It is an advantage of embodiments of the present invention that detection of particular combinations of metabolism analyte concentrations may allow for providing information regarding particular medical conditions or pathologies and/or may allow for improved managing such particular medical conditions or pathologies by controlling lifestyle and dietary conditions. For example, autoimmune diseases are disorders characterised by a disrupted immune behaviour, leading to either a hyper- or hypoactivation of the immune system. Such diseases have specific metabolic chain reactions at the cellular level, leading to a specific immune attack on a specific organ. Globally, the response is an inflammatory response. Inflammation is characterised by increased acidity in the body and a disrupted metabolism.

The health monitoring system may for example be adapted for identifying two or more metabolites of the group of lactate, ketones, ethanol, sugar or pH. The health monitoring system may use such a metabolic fingerprint for deriving information regarding risks for chronic kidney disease or metabolic acidosis by a living creature.

The health monitoring system may for example be adapted for identifying two or more metabolites of the group of lactate, ketones, sugar or pH. The health monitoring system may use such a metabolic fingerprint for deriving information regarding risk for Hashimoto's-Hypothyroidism by a living creature, or when such a disease is already present, for managing the disease by controlling lifestyle and/or dietary requirements having a positive effect thereon.

The health monitoring system may for example be adapted for identifying two or more metabolites of the group of lactate, ketones, surgar, urea or pH. The health monitoring system may use such a metabolic fingerprint for deriving information regarding a user's possible response to certain chemotherapies used for fighting cancer.

The health monitoring system may for example be adapted for identifying two or more metabolites of the group of ketons, sugar or pH. The health monitoring system may use such a metabolic fingerprint for deriving information regarding risks for neurodegenerative diseases such as for example Alzheimer and Parkinson's disease, or when such a disease is already present, for managing the disease by controlling lifestyle and/or dietary requirements having a positive effect thereon. The health monitoring system may for example combine such a metabolic fingerprint with other information regarding the user, such as for example age of the user, when evaluating the risk to such neurodegenerative diseases.

The health monitoring system may for example be adapted for identifying two or more metabolites of the group of glucose, pyruvate or lactate. The health monitoring system may use such a metabolic fingerprint for deriving information regarding risks for Lupus by a user, or when such a disease is already present, for managing the disease by controlling lifestyle and/or dietary requirements having a positive effect thereon.

The health monitoring system may for example be adapted for identifying two or more metabolites of the group of glucose, lactate or urea. The health monitoring system may use such a metabolic fingerprint for deriving information regarding risks for Rheumatoid Arthritis by a user, or when such a disease is already present, for managing the disease by controlling lifestyle and/or dietary requirements having a positive effect thereon.

The health monitoring system may for example be adapted for identifying two or more metabolites of the group of urea, uric acid, lactate dehydrogenase or fasting glucose. The health monitoring system may use such a metabolic fingerprint for deriving information regarding risks for major depressive disorder by a user, or when such a disease is already present, for managing the disease by providing recommendations for controlling or positively influencing lifestyle and/or dietary requirements. Such recommendations may be data-driven recommendations provided by the continuous metabolite monitoring system.

Additionally, microbiota may also be used as metabolism analytes for certain conditions or diseases such as for example for obesity, autoimmune conditions, inflammatory bowel disease, cancer or neurodegeneration disorders.

The health monitoring system may for example be adapted for identifying for example creatinine and pH for deriving information regarding risks for decompensation hart failing or kidney failing.

The health monitoring system may for example be adapted for identifying albumin for evaluating risks of dehydration, but also for identifying risk to malnutrition, significant liver disease, renal loss e.g. in nephrotic syndrome, hormone therapy and pregnancy.

According to embodiments of the present invention, the health monitoring system may be adapted for obtaining information regarding the user's medical condition.

The medical condition may comprise information obtained from an electronic patient file, obtained directly from the user, obtained from a medical practitioner, obtained based on medical interventions, obtained from medical examination, etc.

Information from health monitoring systems of different users may be shared at a central server, optionally anonymised. It is an advantage of embodiments of the present invention that such a system may allow for gathering information from a large group of users. Based on the information obtained, big data analysis may be performed and correlations between certain metabolism analyte fingerprints and medical conditions or certain pathologies may be identified, which can be used for future health care optimisation. It is an advantage of embodiments of the present invention that in big data analysis predetermined algorithms, artificial intelligence including neural networks, self-learning algorithms and alike may be used for identifying such correlations.

The system may be configured to measure the different metabolism analytes at different moments in time.

The health monitoring system may be adapted for measuring the metabolism analytes continuously, quasi-continuously or at predetermined intervals over time.

The system may be configured to, based on said determining for each of the plurality of metabolism analytes whether the obtained concentration information is within a predetermined range, trigger additional measurements of one or more metabolism analytes or to alter a frequency of measurement of one or more metabolism analytes.

It is an advantage of embodiments of the present invention that dynamic adjustment of the health monitoring can be performed, thus resulting in a more personalised health monitoring and optionally continuous health monitoring and thus allowing better control and management of the health for a specific user.

According to embodiments of the present invention, the system may be configured for, based on said determining for each of the plurality of metabolism analytes whether the obtained concentration information is within a predetermined range, providing an output to a user, to a medical practitioner, to a medical team or to other interested parties. The health monitoring device therefore may comprise an output means, e.g. output port, which may for example be implemented in the external device, e.g. in a dedicated device or in a dedicated computer program application. Based on the processed data, personalised recommendations can be made for potential interventions for driving and improving the health of the user. Such personalised recommendations may be based on collected personal and/or global information.

Some examples of personalised recommendations may be related to dietary adaptions for example be whether it is ok or not ok to eat, whether certain food can be allowed at present or not, whether medical drugs should be taken or not taken at present or at which moment such intake should be performed, whether to focus on more protein rich or carb-rich or fat rich food, whether stress management would help or not the current condition . . . , whether certain physical activity can be performed at present or not, etc.

Output also may include providing the user with a warning for consulting a medical practitioner or for providing a warning to a medical practitioner of the user, with providing timing information for scheduling a further consult with a medical practitioner, advising certain medical examination, . . . The output also may comprise merely data information regarding the obtained concentration information of the plurality of metabolism analytes, for displaying warnings regarding the measured metabolism analytes, for indicating deviations of the measured metabolism analytes with respect to predetermined ranges, and alike.

The health monitoring system may be equipped with an auditive or display system for displaying information as identified above.

According to embodiments of the present invention, the health monitoring system further may comprise an input port for obtaining information regarding one or more physiological parameters of the living creature at the time of measurement of the plurality of metabolism analytes in bodily tissue or bodily fluids of the living creature.

The physiological parameter may be one or more of a temperature, information regarding the heart rate such as for example a heart rate variability, blood pressure, motion-based activity, food uptake, tissue conductivity, brain activity such as for example alpha, beta, gamma, delta-wave patterns, respiratory rate, oxygen or carbon monoxide saturations, physical activity, sleep pattern, menstruation cycle, stress, agenda, etc.

In some embodiments, the system may be adapted for obtaining the input from a user by manual input, auditive input, tactic input, etc. And the system may be equipped with a graphical user interface.

The input port of the health monitoring system may be configured for receiving the information regarding one or more physiological parameters from a wearable, worn by the living creature.

Such a wearable may comprise a temperature sensor, a heart pulse rate sensor, a motion sensor, a sensor configured for identifying food uptake, a blood pressure sensor, temperature, information regarding the heart rate such as for example a heart rate variability, blood pressure, motion-based activity, food uptake, tissue conductivity, brain activity such as for example alpha, beta, gamma, delta-wave patterns, respiratory rate, oxygen or carbon monoxide saturations, physical activity, sleep pattern, agenda, position, weather conditions, etc . . .

It is an advantage of embodiments of the present invention that a good synchronisation between the measured metabolism analyte concentration information and the physiological parameter information is obtained, thus allowing more accurate correlation between measured metabolism analyte concentration information and events such as food uptake, stress, activity, and alike.

The health monitoring system is configured for receiving user input. The health system may be adapted for receiving user input, e.g. manual user input, such as information regarding a food uptake event which may for example be combined with information regarding a blood sugar level, information regarding any of the above identified activities or conditions.

The health monitoring system may be adapted for prompting the user for input when one or more metabolism analyte concentrations outside the predetermined range is detected.

It is an advantage of embodiments of the present invention that user input may be prompted for when a detection of a metabolism change is performed by the system. Since the health monitoring system allows for continuous monitoring, upon detection of a change, direct information from the user can be obtained, allowing better corelation between a metabolism change and e.g. an activity or event.

The health monitoring system may be adapted for receiving information regarding the user via user input, via apps used by the user.

The health monitoring system may be configured for deriving from the physiological parameters a stress condition and for identifying that the measured metabolism analyte concentrations are measured under the stress condition. The system may furthermore be triggered for performing further measurement of metabolism analytes at a later moment in time.

It is known that stress influences metabolism through activation and deactivation of specific functions as controlled by the autonomous nervous system during sympathetic or parasympathetic activation. This has an impact on a number of metabolism analytes in the body of a living creature (e.g. whether digestion takes place at its full functionality or not). Concentration information of metabolism analytes obtained under certain stress conditions therefore may provide an image of the metabolic response during a stressful (sympathetic activation) situation, and give information to the metabolic response under a given autonomic activation (sympathetic=stress=fight or flight, parasympathetic=rest & digest). This information can provide input to the actual health condition of the living creature and can trigger appropriate intervention to improve it (e.g. by proactively assisting in parasympathetic activation previous and/or during food ingestion, for example through guided exercises such as breathing). It is an advantage of embodiments of the present invention that a health monitoring system based on an implantable sensor allows for obtaining information at different moment in times. Whereas conventional analysis of metabolism analytes is typically based on sampling e.g. blood from a living creature which typically needs to be done by a medical practitioner and which is an action known to induce stress for several patients, the continuous monitoring system of embodiments of the present invention can obtain information at any moment in time, without—once the implantable sensor has been implanted—the need for intervention of a medical practitioner. Therefore the health monitoring system allows for the possibility of obtaining more thrustworthy metabolism analyte concentration information obtained at a moment in time where the living creature is less subject to the measurement stress and where the impact of direct environmental factors (e.g. stressful environment) can be detected. The latter allows for a more accurate determination of the actual personalized response of the metabolism and thus the health condition of the living creature. More generally, the health monitoring system assists in determining the environmental influence to the metabolic fingerprint at any given situation, understanding better the trends of stress-impact (or stress-absence impact) to how the health of the user responds.

The health monitoring system may comprise an input port for obtaining information regarding one or more of the user's location, hobbies, ethnic background, age, gender, socio-economical status, etc.

The health monitoring system may be adapted for receiving information regarding the user via user input, via apps used by the user.

Information from health monitoring systems of different users may be shared at a central server, optionally anonymised.

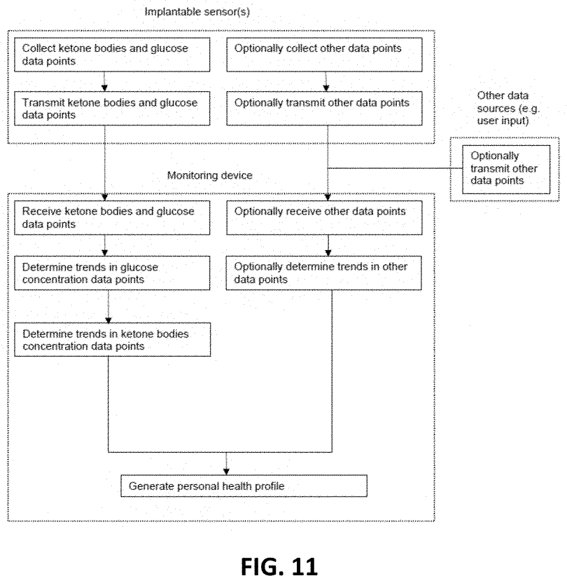

According to one aspect, the present invention also relates to a health monitoring platform, the health monitoring platform comprising a plurality of health monitoring systems as described above for use by a plurality of individual users and further comprising a central processing unit for processing metabolic fingerprint information obtained from the plurality of health monitoring systems. The central processing unit may for example be a remote server system, which is arranged for collecting the metabolic fingerprint information from different users or personal health information from different users, obtained through the plurality of health monitoring systems. In another embodiment, the platform is equipped with an algorithm for deriving information regarding metabolic fingerprints and certain conditions or diseases, for deriving information regarding physiological parameters and metabolic fingerprints, for storing and providing recommendations based on certain measured metabolic fingerprints optionally completed with other information, and alike. The platform may be organised as shown and described in FIG. 11, whereby the data information provided by the individual health monitoring systems comprises metabolic fingerprints and information regarding whether the metabolism analyte concentrations are within predetermined ranges.

It is an advantage of embodiments of the present invention that such a system may allow for gathering information from a large group of users. Based on the information obtained, big data analysis may be performed and correlations between certain metabolism analyte fingerprints and other information such as for example physiological information or other user information. The latter may assist in identifying causes of certain metabolism-based diseases. The latter may for example also be used for specific targeted investigation trials in which the influence of multiple parameters can be studied.

It is an advantage of embodiments of the present invention that predetermined algorithms, artificial intelligence including neural networks, self-learning algorithms and alike may be used for identifying such correlations. It is an advantage of embodiments of the present invention that in big data analysis predetermined algorithms, artificial intelligence including neural networks, self-learning algorithms and alike may be used for identifying such correlations.

Illustrative examples are provided herein to demonstrate principles and advantages of embodiments according to the present invention. These examples are not intended to be limiting to the invention in any way, but merely provided to assist the skilled person in reducing embodiments of the present invention to practice.

In one aspect, the present invention also relates to methods for obtaining personal health information for an individual by performing the steps corresponding with the functionality of the different components of the personal health monitoring system described above. According to some embodiments, a method for obtaining personal health information for an individual is disclosed, the method comprising

simultaneously measuring a plurality of metabolism analytes in bodily tissue or bodily fluids of the living creature, thus obtaining a metabolic fingerprint for a living creature comprising concentration information of the plurality of metabolism analytes at any given moment in time, and determining for each of the plurality of metabolism analyte whether the obtained concentration information is within a predetermined range. In some embodiments, the method may comprise providing an output to a user, a medical practitioner or a medical team, the output comprising personalised recommendations. The health monitoring system as described in embodiments of the present invention may be programmed for performing the method steps as described above, e.g. programmed with a corresponding algorithm.

In one aspect, the present invention discloses a personal health monitoring system, with an implantable sensor for sensing at least glucose concentration data points and ketone bodies concentration data points and a monitoring device comprising a processor for determining trends in the glucose concentration data points and trends in the ketone bodies concentration data points for generating a personal health profile of the user.

The fatty acid metabolism and the glucose metabolism are two groups of biochemical processes which are responsible for most of the energy generation and consumption in mammals. They are also responsible for the formation, breakdown and interconversion of biologically important molecules.

The measurement of a fat metabolism analyte, such as ketone bodies, is indicative of fat metabolism.

Ketosis in humans is a nutritional process characterised by serum concentrations of ketone bodies over 0.5 mM, with low and stable levels of insulin and blood glucose. Long-term ketosis may result from fasting or staying on a low-carbohydrate diet (ketogenic diet), and deliberately induced ketosis can be a lifestyle choice or be used as a medical intervention for various conditions, such as intractable epilepsy, and the various types of diabetes. Ketosis can also occur in animals, for example in dairy cattle during the first weeks after giving birth to a calf or to sheep in pregnancy toxemia.

Ketoacidosis is a pathological metabolic state marked by extreme and uncontrolled ketosis caused by, for example, alcohol, starvation or diabetes. In ketoacidosis, the body fails to adequately regulate ketone production causing such a severe accumulation of keto acids that the pH of the blood is substantially decreased, eventually leading to coma and death.

Monitoring ketone bodies in a mammal is thus expected to provide essential information for subjects at risk of ketosis because of medical, dietary or lifestyle conditions.

The measurement of a glucose metabolism analyte, such as glucose, is indicative of glucose metabolism.

Hypoglycaemia, also known as low blood sugar, is a condition characterized by blood sugar levels below normal levels. This may result in a variety of symptoms including clumsiness, trouble talking, confusion, loss of consciousness, seizures, or death. A feeling of hunger, sweating, shakiness, and weakness may also be present. Hypoglycaemia may be present as a consequence of medical conditions (such as diabetes), a side-effect of a medical treatment, dietary or lifestyle conditions.

Hyperglycaemia, also known as high blood sugar, is a condition characterized by blood sugar levels above normal levels. Acute hyperglycaemia can result in polyuria, polydipsia, weight loss, sometimes with polyphagia, and blurred vision and chronic hyperglycaemia may result in a range of medical conditions such as kidney damage, neurological damage, cardiovascular damage, damage to the retina, feet and legs. Hyperglycaemia may be present as a consequence of medical conditions (such as diabetes), a side-effect of a medical treatment, dietary or lifestyle conditions.

2 hour plasma glucose (2hPG), fasting plasma glucose (FPG), random plasma glucose (PG) are widely used markers of glycemic control. Continuous glucose monitoring is the prerequisite to enable strict glycemic control, keeping blood glucose levels within a desired range, such as a range that prevents medical complications. The desired range is highly personal and may be governed by factors such as medical conditions, dietary of lifestyle choices. Even within the commonly accepted “healthy” range of 80-110 mg/dl blood glucose, the ideal blood glucose level for every individual is different.

Embodiments of the invention provide a personal health monitoring system, comprising an implantable sensor and a monitoring device, and capable of generating an improved personal health profile from the collected sensor data.

The health monitoring system may provide insight in the metabolic state of a user, by providing user dependent correlations between ketone bodies and glucose levels. The analysis of trends in glucose and ketone bodies levels while taking into account personal correlations may provide improved means for subjects to manage their metabolic state. For example, for patients suffering from diabetes, the change from modest hyperglycaemia to ketoacidosis can occur slowly or very rapidly, depending on the type of diabetes and the individual patient (e.g. infants vs. adults). Improved analysis of trends and prediction, taking into account individual correlations between ketone bodies and glucose levels, may be of vital importance for these patients. Patients following a ketogenic diet as (complementary) treatment of brain tumor may also benefit from the user dependent correlations between ketone bodies and glucose levels which are the object of some preferred embodiments of the personal health monitoring system of the present invention.

The ability to maintain blood glucose and ketone bodies within a desired range requires frequent measurements of glucose and ketone bodies. Each ketone bodies and glucose measurement provides information about the fatty acid and glucose metabolism that can be used to determine the personal health profile of a subject. This subject may be a patient (e.g. suffering from diabetes) or any person seeking to monitor and improve their personal health profile.

The implantable sensor of the health monitoring system of the present invention is shown in FIG. 1 and comprises sensor housing 1, means for sensing biological parameters in bodily fluids 2, an area of sensor housing which is adapted to the sensing means 3 and a wireless transceiver 4. The implantable sensor may be capable of sensing biological parameters based on surface chemical reactions and/or by using optical means. The implantable sensor may be capable of performing a reagent-free optical analysis method. The implantable sensor may comprise biocompatible packaging in order to reduce or minimize the risk of bio-fouling.

A preferred embodiment of an implantable sensor is shown in FIG. 2 and is equipped with optical sensing means 5 and an optical processor 6 (e.g. a single-chip optical sensor) for continuous analyte monitoring. The implantable sensor comprises an advanced optical processor in the sensor, allowing advanced and optionally complex radiation processing, e.g. allowing spectral and depth-resolved processing of radiation received or guided to a measurement region.

The implantable sensor comprises means for sensing biological parameters in bodily fluids and comprises a wireless transceiver for transmitting sensor data containing data points which are provided by said sensing means upon sensing biological parameters. The implantable sensor is provided for measuring at least glucose and ketone bodies in bodily fluids. The sensing means may be as described in U.S. Pat. No. 9,532,738 B2, in particular column 11 line 15-67, which are hereby incorporated by reference.

In embodiments of the invention, the implantable sensor may be adapted for sensing biological parameters in bodily fluids wherein the bodily fluid may be interstitial fluid, ocular fluid, intermuscular fluid or peritoneal fluid. It has been found that measurements of biological parameters in the interstitial fluid present a reliable relationship with blood values, are minimally invasive and safe and present other advantages such as the elimination of the need for anticoagulants. Thus, in a preferred embodiment of the invention, the implantable sensor is a subcutaneous implantable sensor.

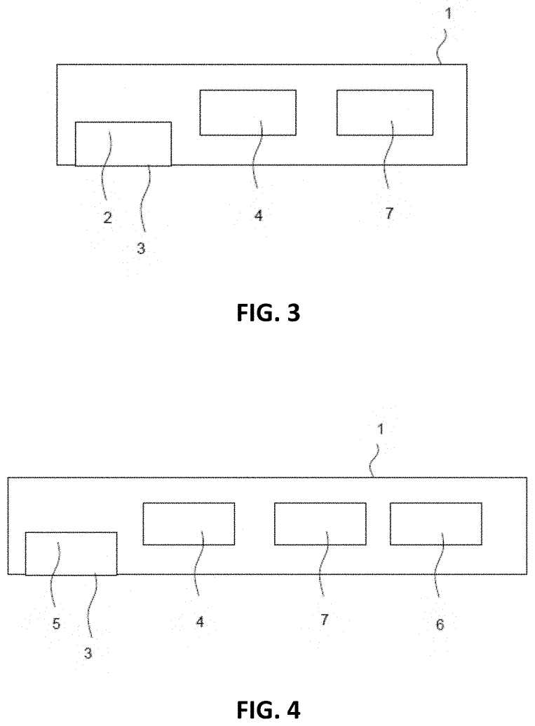

In embodiments of the invention, the implantable sensor may comprise a rechargeable battery and/or components for wireless energy transfer. A preferred embodiment of an implantable sensor is shown in FIG. 3 and is equipped with components for wireless energy transfer 7. A more preferred embodiment of an implantable sensor is shown in FIG. 4 and is equipped with optical sensing means 5 and an optical processor 6 and components for wireless energy transfer 7. In a preferred embodiment, the rechargeable battery is a solid state battery.

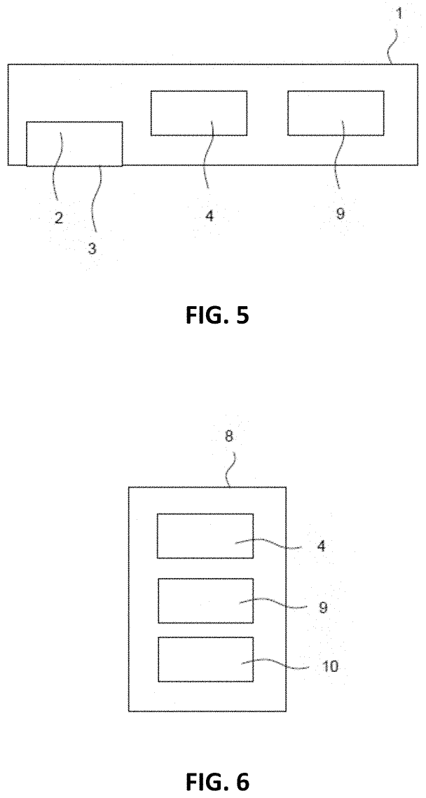

In embodiments of the invention, the implantable sensor may further be equipped with means for processing sensor data, wherein said processing means is equipped with an algorithm which is executable on said processing means and which is provided for converting sensor data before transmitting to the monitoring device. A preferred embodiment of an implantable sensor is shown in FIG. 5 and is equipped with a processing means 9.

The implantable sensor may further be capable of sensing heart rate, body temperature, urea, lactate, pH, fructosamine, oxaloacetate and/or hydration level.

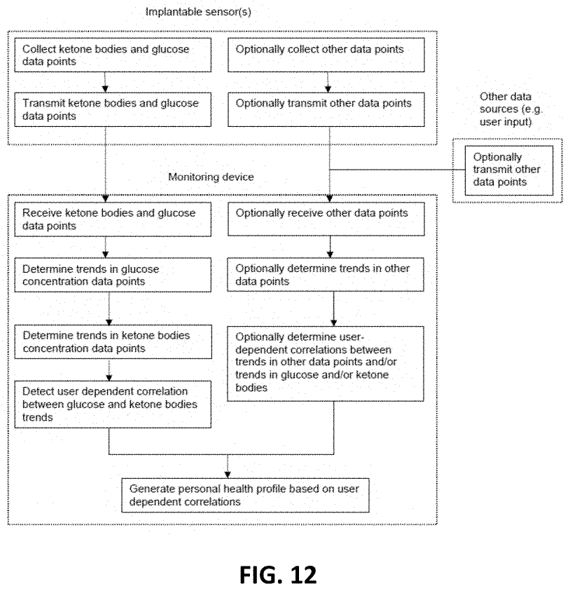

A preferred embodiment of a monitoring device is shown in FIG. 6 and comprises a housing 8, a wireless transceiver 4 for communicating with the wireless transceiver of the implantable sensor or other devices of the monitoring device to receive sensor data. The monitoring device further comprises processing means 9 for processing said sensor data and a memory 10 for storing data. The processing means is equipped with an algorithm, an embodiment of which is shown in FIG. 11, which is loadable into the memory 10 for execution by said processing means. The algorithm is at least provided for: determining trends in glucose concentration data points and in ketone bodies concentration data points and generating a personal health profile of the user. In embodiments according to the invention, shown in FIG. 12, the algorithm of the monitoring device may be provided for performing the following steps: determining first trends in said glucose concentration data points and second trends in said ketone bodies concentration data points; detecting first user dependent correlations between said first trends and said second trends, and generating a personal health profile of the user based on said first user dependent correlations. In a preferred embodiment of the invention the monitoring device is a smartphone. In another preferred embodiment of the invention the monitoring device is a smart watch.

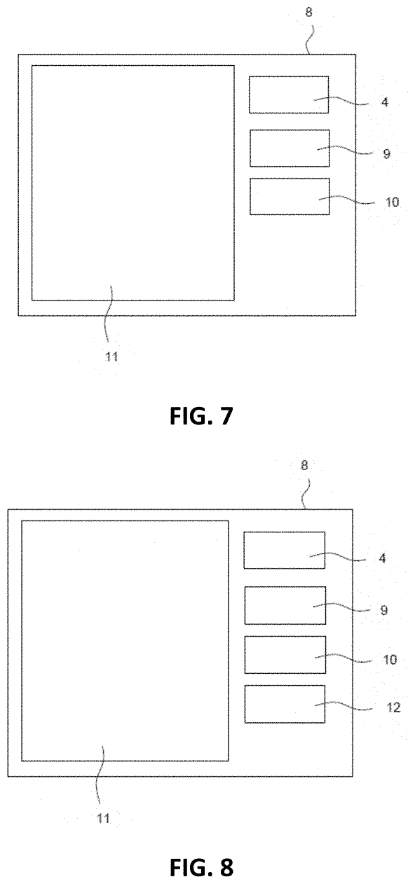

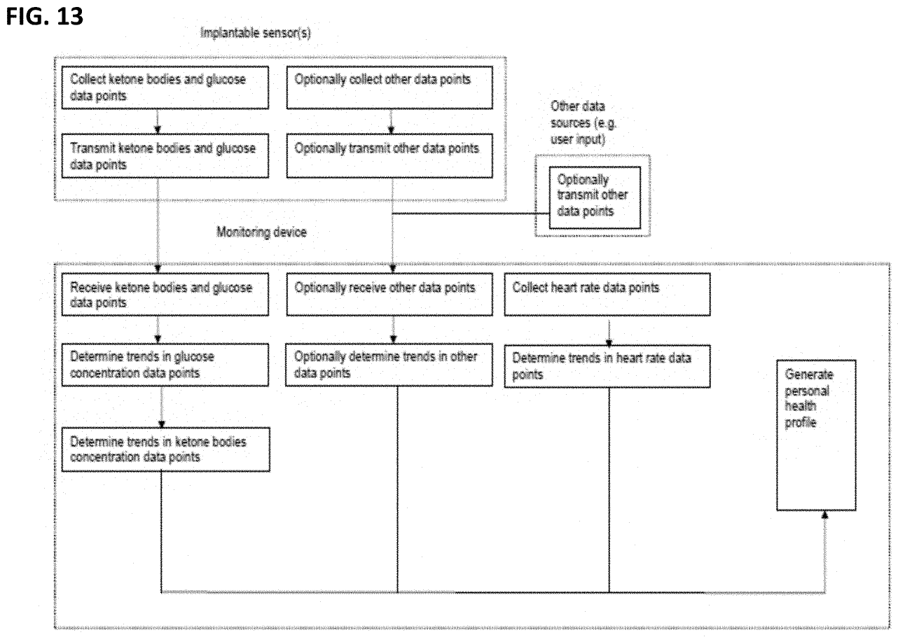

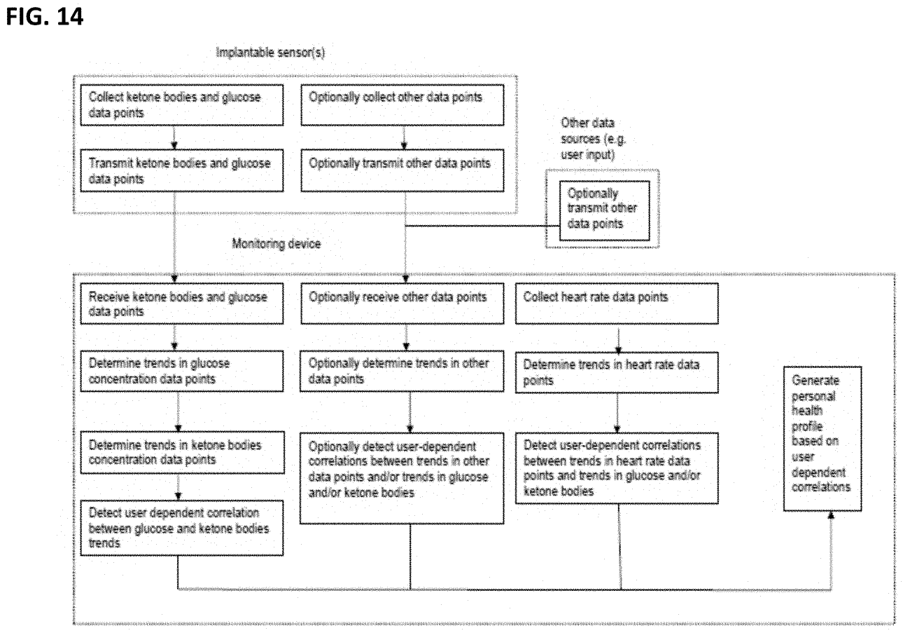

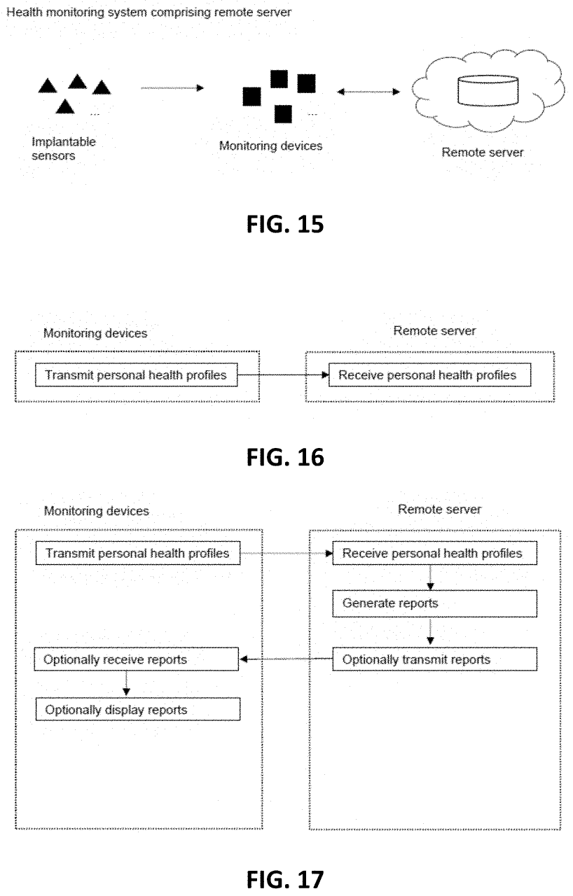

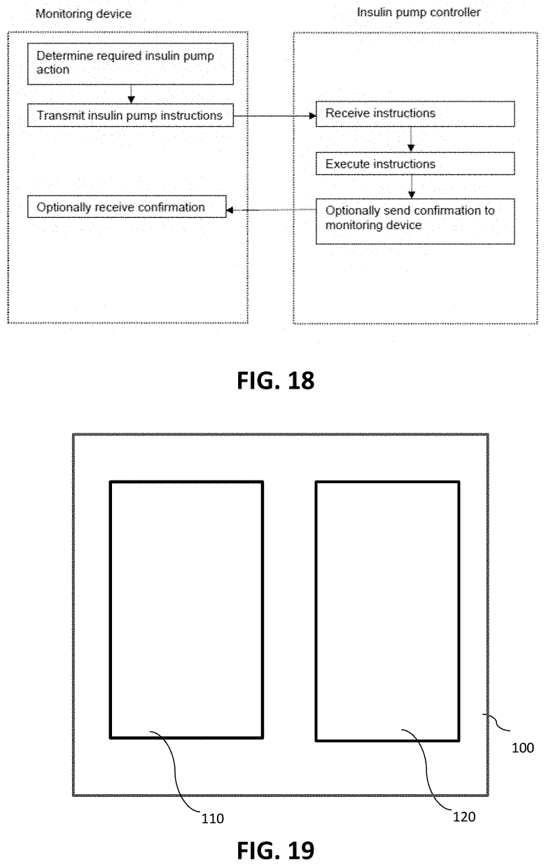

Another embodiment of the monitoring device is shown in FIG. 7 wherein the monitoring device is equipped with a user interface, comprising a display 11 for displaying the personal health profile and interacting with the user. The monitoring device may further be provided for receiving heart rate data points from a heart rate sensor, said heart rate data points being received from the implantable sensor or any other device which is capable of providing heart rate data points. For example, in case the monitoring device is a smart watch, a heart rate sensor may be provided on board the smart watch. FIG. 8 shows a preferred embodiment of the monitoring device wherein the monitoring device is equipped with a heart rate sensor 12. The algorithm may be provided for determining trends in the heart rate data points and detecting user dependent correlations between trends in heart rate data points and trends in glucose concentration data points and/or ketone bodies concentration data points, and evaluating said user dependent correlations upon generating a personal health profile. Thus, a further improved personal health profile may be achieved. FIGS. 11 and 12 show embodiments of the algorithm wherein the heart rate data points could be received as other data points, while FIGS. 13 and 14 show embodiments of the algorithm wherein the heart rate data points are being collected by the monitoring device.

In another embodiment of the present invention the monitoring device may further be provided for receiving and processing data points of one or more of the following parameters: body temperature, urea, lactate, pH, fructosamine, oxaloacetate and/or hydration level. These parameters may be provided by the implantable sensor or any one or more additional implantable sensors and/or other device which is capable of providing data points of one or more of said parameters. The algorithm executable on the processing means may then be provided for determining trends in the data points of the one or more additional parameters (body temperature, urea, lactate, pH, fructosamine, oxaloacetate and/or hydration level) and generating a personal health profile. In a preferred embodiment the algorithm executable on the processing means may then be provided for determining trends in the data points of the one or more additional parameters (body temperature, urea, lactate, pH, fructosamine, oxaloacetate and/or hydration level), detecting user dependent correlations between said trends and trends in heart rate data points, trends in glucose concentration data points and/or ketone bodies concentration data points, and evaluating said user dependent correlations upon generating a personal health profile. Thus, a further improved personal health profile may be achieved. FIGS. 11 and 12 show embodiments of the algorithm wherein the body temperature, urea, lactate, pH, fructosamine, oxaloacetate and/or hydration level data points could be received as other data points.

In another embodiment of the present invention the monitoring device may further be provided for receiving and processing data points of one or more of the following parameters: nutritional intake such as carbohydrate intake data points, activity such as accelerometer data points and/or blood pressure data points and location such as GPS data points, agenda item data points. These parameters may be provided by the implantable sensor or any one or more additional implantable sensors and/or other device which is capable of providing data points of one or more of said parameters and/or manual user input. The algorithm executable on the processing means may then be provided for determining trends in the data points of the one or more additional parameters (nutritional intake, activity, location) and generating a personal health profile. In a preferred embodiment the algorithm executable on the processing means may then be provided for determining trends in the data points of the one or more additional parameters (nutritional intake, activity), detecting user dependent correlations between said trends and trends in heart rate data points, trends in glucose concentration data points and/or ketone bodies concentration data points, and evaluating said user dependent correlations upon generating a personal health profile. Thus, a further improved personal health profile may be achieved. FIGS. 11 and 12 show embodiments of the algorithm wherein the nutritional intake, activity, location could be received as other data points.

The glucose ketone index is a biomarker that refers to the molar ratio of circulating glucose over β-OHB, which is the major circulating ketone body. The glucose ketone index is a single value that can assess the relationship of the glucose to ketone bodies. The glucose ketone index is described in Meidenbauer et al. Nutrition & Metabolism 2015, 12:12, which is incorporated herein by reference. In another embodiment of the present invention, the monitoring device may comprise an algorithm executable on the processing means provided for determining the glucose ketone index (GM). In a preferred embodiment the algorithm executable on the processing means may then be provided for determining trends in the glucose ketone index, detecting user dependent correlations between said trends and trends in heart rate data points, trends in glucose concentration data points and/or ketone bodies concentration data points, and evaluating said user dependent correlations upon generating a personal health profile. Thus, a further improved personal health profile may be achieved.

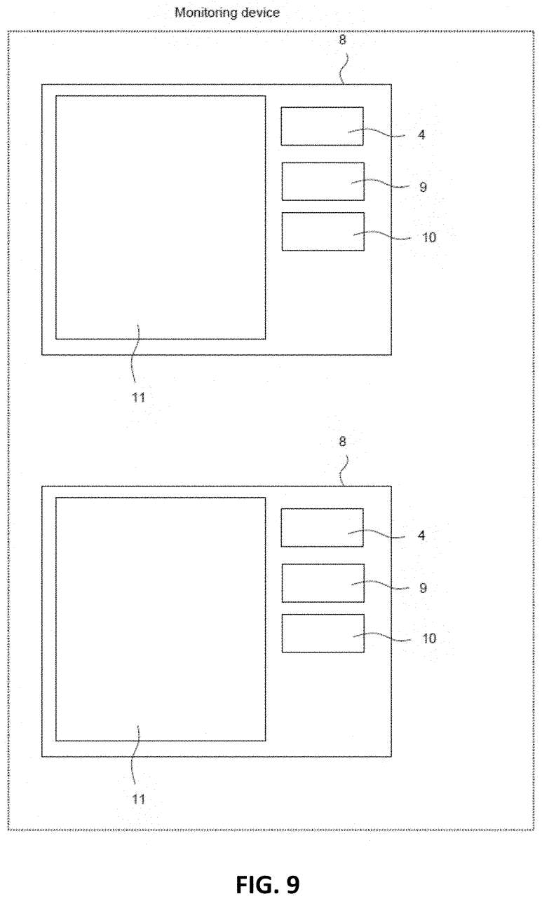

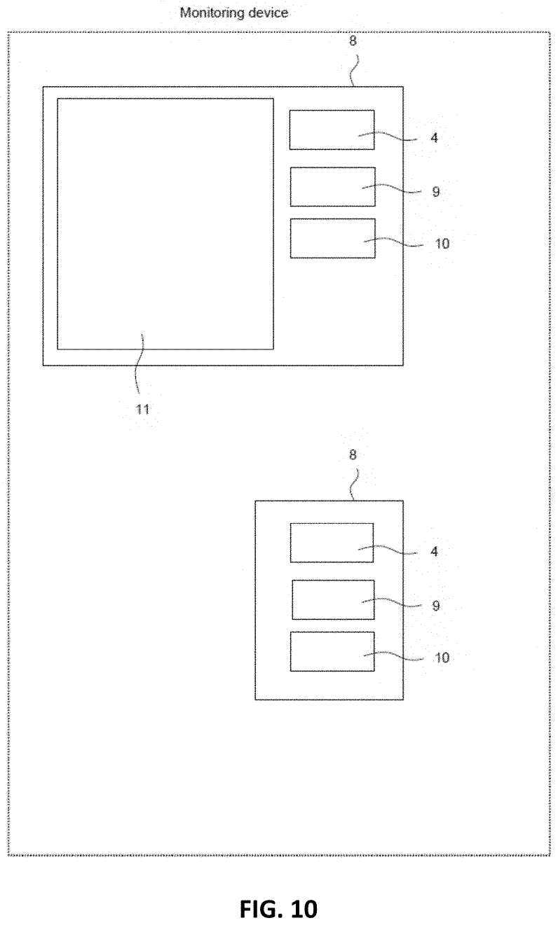

In another embodiment of the invention, different features of the monitoring device may be present on different devices. The monitoring device may thus be an ensemble of two or more devices, each device comprising a transceiver for receiving and transmitting data. FIGS. 9 and 10 show an embodiment of the invention wherein the monitoring device is an ensemble of devices. In a preferred embodiment the monitoring device comprises one or more of the following devices: a device equipped with a transceiver and a processor, a smartphone and a cloud server. In a more preferred embodiment the device equipped with a transceiver and a processor is capable of transmitting and receiving at least two different communication protocols. In embodiments, the at least two different communication protocols may be wired and/or wireless signals. In embodiments, the wireless signals may comprise signals according to different wireless bands and/or protocols such as IEEE802.11, bluetooth, cellular etc.

In another embodiment of the invention, the monitoring device may be an ensemble of two or more devices, each device comprising a transceiver for receiving and transmitting data, wherein two or more devices are each equipped with a processing means and an algorithm executable on the processing means. In a preferred embodiment of the invention, the algorithm of each device is provided performing one or more steps necessary for generating a personal health profile according to the invention.

In embodiments the monitoring device may be provided for generating and/or communicating to the user personalized behavioural, lifestyle and therapeutic suggestions and actions, based on the monitoring of biological parameters. In embodiments, the monitoring device may be provided for generating instructions for a controller of an insulin pump or may form part of an insulin pumping device.

In embodiments the monitoring device may be an ensemble of two or more devices wherein each device comprising a transceiver for receiving and transmitting data, wherein two or more devices are each equipped with a processing means and an algorithm executable on the processing means wherein the algorithm of each device is provided for performing one or more steps necessary for generating and/or communicating to the user personalized behavioural, lifestyle and therapeutic suggestions and actions, based on the monitoring of biological parameters. In a preferred embodiment of the invention the monitoring device is an ensemble of devices which includes an insulin pumping device and/or controller of an insulin pump. In a more preferred embodiment of the invention the monitoring device is an ensemble of devices which includes an insulin pumping device and/or controller of an insulin pump and a smartphone.

In a preferred embodiment of the invention, the monitoring device is an ensemble of devices which includes a remote server. In a more preferred embodiment of the invention, the monitoring device is an ensemble of devices which includes a remote server wherein the remote server is equipped with algorithm which is executable on said remote server and provided for performing one or more steps necessary for generating and/or communicating to the user a personal health profile and/or personalized behavioural, lifestyle and therapeutic suggestions and actions, based on the monitoring of biological parameters. In another preferred embodiment of the invention, the monitoring device is an ensemble of devices which does not include a remote server.

A preferred embodiment of the present invention, shown in FIG. 11 provides a multiple user health monitoring system comprising a plurality of personal health monitoring systems and further comprising a remote server system which is provided for collecting the personal health profiles generated by the plurality of personal health monitoring systems. In another embodiment, the remote server system is equipped with a further algorithm which is executable on said remote server system and provided for generating reports based on said collected personal health profiles. The reports may include personal recommendations optionally sent back to the monitoring device and displayed to the user, anonymous statistical data from a group of users or instructions for other devices such as an insulin pump. The reports may also include instructions for the monitoring device to be relayed to the implantable sensor.

The invention also provides a method to generate a personal health profile comprising measuring glucose concentration data points and ketone bodies concentration data points using an implantable sensor, transmitting glucose concentration data points and ketone bodies concentration data points to a monitoring device, determining first trends in said glucose concentration data points and second trends in said ketone bodies concentration data points, detecting first user dependent correlations between said first trends and said second trends, and generating a personal health profile of the user based on said first user dependent correlations.

In embodiments the monitoring device may be provided for generating and/or communicating to the user personalized behavioural, lifestyle and therapeutic advice and/or interventions, based on the monitoring of biological parameters. In a preferred embodiment of the present invention the behavioural, lifestyle and therapeutic suggestions and actions may be one or more of the following: nutritional advice, emergency care advice, therapeutic advice and/or interventions, insulin pump controller instructions.

### EXAMPLES

Embodiments of the invention may for example be provided for detecting and correlating trends as follows.

Useful glucose and ketone concentration trends to be detected are for example:

Moderate ketones and hypoglycaemia resulting in the generation of a personal health profile and recommendations and/or instructions comprising: increase carb intake and/or decrease basal insulin administration.

High ketones and hyperglycaemia resulting in the generation of a personal health profile and recommendations and/or instructions comprising: possible insulin pump defect, danger for ketoacidosis, drink water and consult emergency medical care.

Low ketones and hypoglycaemia resulting in the generation of a personal health profile and recommendations and/or instructions comprising: possible insulin overadministration or insulin pump defect, increase carb intake.

Linking glucose and ketone trends with glucose variability (GV) and HbAlc levels to generate a personal risk and health profile for complications of diabetes. This will enable the patient to improve his/her risk profile.

Group analysis of glucose and ketones trend correlations enabling better prediction and treatment models based on patient type (age, gender, BMI, physical activity level).

Useful heart rate trends to be detected are for example:

Loss in heart rate variability in combination with progressive hypoglycaemia resulting in the generation of a personal health profile and recommendations and/or instructions comprising: prevention of possible severe hypoglycaemia, consult emergency care.

Useful user dependent correlations are for example:

Known high response rate of ketones and glucose to insulin administration resulting in the generation of a personal health profile and recommendations and/or instructions comprising: adapting the amount of insulin to be administered accordingly.

1. A health monitoring system for monitoring health of a living creature, the health monitoring system comprising:
an implantable sensor for simultaneously measuring a plurality of metabolism analytes in bodily tissue or bodily fluids of the living creature, thus obtaining a metabolic fingerprint for a living creature comprising concentration information of the plurality of metabolism analytes at any given moment in time;
a processor programmed for determining for each of the plurality of metabolism analyte whether the obtained concentration information is within a predetermined range.

2. The health monitoring system according to claim 1, wherein the processor furthermore is programmed for deriving from said determination a homeostatic and/or alleostatic condition of the living creature.

3. The health monitoring system according to claim 1, wherein the plurality of metabolism analytes comprises at least three different metabolism analytes.

4. The health monitoring system according to claim 1, wherein the plurality of metabolism analytes is a selection of two or more of glucose, ketones, lactate, lactic acid, acetic acid, nitrite, creatinine, hormones, vitamine B11, vitamine B12, acetate, propionic acid, propionate, butyric acid, butyrate, phenyl sulfate, urea, pyruvate, ethanol, sugar, pH, uric acid, lactate dehydrogenase, fasting glucose, sodium, potassium or cholesterol.

5. The health monitoring system according to claim 1, wherein the health monitoring system is adapted for obtaining information regarding the user's medical condition.

6. A health monitoring system according to claim 1, wherein the health monitoring system furthermore comprises an output port for providing an output to a user, a medical practitioner or a medical team and wherein the health monitoring system is programmed for providing personalised recommendations to the user through the output port.

7. The health monitoring system according to claim 1, wherein the system is configured to measure the different metabolism analytes at different moments in time.

8. The health monitoring system according to claim 7, wherein the system is configured to, based on said determining for each of the plurality of metabolism analytes whether the obtained concentration information is within a predetermined range, trigger additional measurements of one or more metabolism analytes or to alter a frequency of measurement of one or more metabolism analytes.

9. The health monitoring system according to claim 1, wherein the system is configured for, based on said determining for each of the plurality of metabolism analytes whether the obtained concentration information is within a predetermined range, providing the user with a warning for consulting a medical practitioner or for providing a warning to a medical practitioner of the user.

10. The health monitoring system according to claim 1, the health monitoring system further comprising an input port for obtaining information regarding one or more physiological parameters of the living creature at the time of measurement of the plurality of metabolism analytes in bodily tissue or bodily fluids of the living creature.

11. The health monitoring system according to claim 10, wherein the input port of the health monitoring system is configured for receiving the information regarding one or more physiological parameters from a wearable, worn by the living creature.

12. The health monitoring system according to claim 10, wherein the health monitoring system is configured for receiving user input from a user, optionally through a graphical user interface, or
wherein the health monitoring system is programmed for prompting the user for input when one or more metabolism analyte concentrations outside the predetermined range is detected.

13. The health monitoring system according to claim 10, wherein the health monitoring system is configured for deriving from the physiological parameters a stress condition and for identifying that the measured metabolism analyte concentrations are determined under the stress condition.

14. The health monitoring system according to claim 1, wherein the health monitoring system comprises an input port for obtaining information regarding one or more of the user's location, hobbies, ethnic background, age, gender, socio-economical status, etc.

15. The health monitoring system according to claim 1, wherein the processor is configured for comparing with predetermined ranges for the metabolism analyte concentrations, whereby the predetermined ranges are based on previously measured metabolism analyte concentrations from the living creature.

16. The health monitoring system according to claim 1, wherein the processor is configured for comparing with predetermined ranges for the metabolism analyte concentrations, whereby the predetermined ranges are based on previously measured metabolism analyte concentrations obtained from a group of living creatures.

17. The health monitoring system according to claim 16, wherein the system is adapted for obtaining information regarding to any of gender, age, ethnicity or body mass index and wherein the processor furthermore is configured for comparing with predetermined ranges that are based on previously measured metabolism analyte concentrations obtained from a group of living creatures having a same gender, age, etc.

18. The health monitoring system according to claim 1, wherein the implantable sensor comprises an optical sensor configured for spectroscopic measurement of the plurality of metabolism analytes in bodily tissue or bodily fluids.

19. The health monitoring system according to claim 1, the system furthermore comprising an impedance spectroscopy sensor for deriving a hydration status.

20. A health monitoring platform, the health monitoring platform comprising a plurality of health monitoring systems according to claim 1 for use by a plurality of individual users and the health monitoring platform further comprising a central processing unit for processing metabolic fingerprint information obtained from the plurality of health monitoring systems.
